(19) United States
(12) Patent Application Publication
(10) Pub. No.: US 2021/0068778 A1
(43) Pub. Date: Mar. 11, 2021

(54) BIOPSY DEVICES AND METHODS OF USE THEREOF

(71) Applicant: Covidien LP, Mansfield, MA (US)

(72) Inventors: Todd M. Morley, Woodland Park, CO (US); Kasey A. Grim, Boulder, CO (US); Joe D. Sartor, Longmont, CO (US)

(21) Appl. No.: 17/012,800

(22) Filed: Sep. 4, 2020

Related U.S. Application Data
(60) Provisional application No. 62/896,693, filed on Sep. 6, 2019.

(51) Int. Cl. A61B 8/08; A61B 10/02; A61B 8/00; G06T 7/00

(52) U.S. Cl. CPC A61B 8/0841; A61B 10/0275; A61B 8/4477; A61B 8/5207; G06T 2207/30096; A61B 8/463; A61B 8/085; G06T 2207/10132; G06T 7/0012

(57) ABSTRACT

A tissue biopsy system includes a biopsy device, a display, and a processing unit in communication with the biopsy device and the display. The biopsy device includes a handle body, a transducer, and a needle. The transducer is disposed within the handle body. The needle is configured to be deployed from the handle body and into tissue. The display is configured to display an ultrasound image of the tissue based on data collected by the transducer. The processing unit is configured to identify an area of interest, located in the tissue, based on the ultrasound image and visually indicate the area of interest on the display.

### CROSS REFERENCE TO RELATED APPLICATIONS

The present claims the benefit of and priority to U.S. Provisional Application Ser. No. 62/896,693, filed on Sep. 6, 2019, the entire contents of each of which are incorporated herein by reference.

### BACKGROUND

### Technical Field

The present disclosure relates to biopsy sampling and, more particularly, to biopsy systems, ultrasound devices thereof, and methods for navigating a biopsy needle to a target location using the ultrasound device.

### Description of Related Art

To have the best chance of successfully treating cancer, it is critical to diagnose cancer at an early stage. Various methods are used to identify the existence of abnormalities in tissue prior to a patient being symptomatic. For example, women regularly go for prophylactic mammograms to determine whether there are any early stage tumors developing in their breast tissue. Although mammography is effective at identifying whether a tumor is present, mammography is not capable of differentiating between benign and malignant tumors. Accordingly, upon identifying an abnormality in the tissue, the status of the abnormality needs to be determined using an additional diagnostic technique.

One method to verify whether a tissue is cancerous is to obtain a tissue sample for histological examination through a biopsy of the tissue (e.g., breast tissue) near the lesion. There are a number of devices and methods for performing a biopsy. In some instances, a tumor may be identified using manual palpation of the breast tissue and then a biopsy needle may be positioned over the identified tumor to take a sample of tissue. Another method involves holding an ultrasound probe in one hand while holding the biopsy needle with a second hand and guiding the biopsy needle along the image plane of the ultrasound probe.

### SUMMARY

Provided in accordance with the disclosure is a tissue biopsy system including a biopsy device, a display, and a processing unit in communication with the biopsy device and the display. The biopsy device includes a handle body, a transducer, and a needle. The transducer is disposed within the handle body. The needle is configured to be deployed from the handle body and into tissue. The display is configured to display an ultrasound image of the tissue based on data collected by the transducer. The processing unit is configured to identify an area of interest, located in the tissue, based on the ultrasound image and visually indicate the area of interest on the display.

In aspects, the display may be in operable communication with the processing unit.

In aspects, the display may form a proximal end portion of the handle body.

In aspects, the display may be remote from the handle body and coupled to a proximal end portion of the handle body.

In aspects, in identifying the area of interest, the processing unit may apply at least one radiomic transformation on the ultrasound image, process the transformed image with a segmentation and/or classification algorithm, and visually indicate the area of interest by displaying a line defining a border of the area of interest on the display.

In aspects, the processing unit may be further configured to identify a sub-region of interest within the area of interest.

In aspects, in identifying the sub-region of interest, the processing unit may apply at least one radiomic transformation on the ultrasound image, process the transformed image with a segmentation and/or classification algorithm, and may provide a visual indication of the sub-region of interest. The visual indication of the sub-region of interest may include displaying a line defining a border of the sub-region of interest.

In aspects, the sub-region of interest may be a necrotic sub-region and/or an active sub-region.

In aspects, the visual indication of the sub-region of interest may further include highlighting sub-regions of interest and/or applying discrete shading to each type of sub-region of interest.

In aspects, the processing unit may be further configured to identify a biopsy-target area within the active sub-region and visually indicate the biopsy-target area on the display.

In aspects, the processing unit may be further configured to overlay the visual indication of the sub-region of interest over the ultrasound image on the display.

In aspects, the area of interest may be a tumor.

In accordance with another aspect of the disclosure, a method of performing a tissue biopsy includes: receiving, by a processing unit of a tissue biopsy system, an ultrasound image of tissue; identifying an area of interest based on the ultrasound image; and displaying a first visual indication of the area of interest on a display of the tissue biopsy system.

In aspects, identifying the area of interest may include applying a radiomic transformation on the ultrasound image, and processing the transformed image with a segmentation algorithm and/or classification algorithm.

In aspects, displaying the first visual indication may include displaying a line defining a border of the area of interest.

In aspects, the method may further include identifying a sub-region of interest within the area of interest and displaying a second visual indication of the sub-region of interest on the display.

In aspects, identifying the sub-region of interest within the area of interest may include applying a radiomic transformation on the ultrasound image, and processing the transformed image with a segmentation algorithm and/or classification algorithm.

In aspects, the sub-region of interest within the area of interest may be a necrotic sub-region and/or an active sub-region.

In aspects, displaying the first visual indication may include displaying a line defining a border of the area of interest, and displaying the second visual indication may include displaying a line defining a border of the sub-region of interest.

In aspects, displaying the second visual indication may include highlighting the sub-region of interest and/or applying discrete shading to each type of sub-region of interest.

In aspects, the method may further include overlaying the first visual indication and the second visual indication over the ultrasound image.

In aspects, the area of interest may be a tumor.

As used herein, the term “distal” refers to the portion that is being described which is further from a user, while the term “proximal” refers to the portion that is being described which is closer to a user. Further, to the extent consistent, any of the aspects and features detailed herein may be used in conjunction with any or all of the other aspects and features detailed herein.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Various aspects and features of the present disclosure are described hereinbelow with references to the drawings, wherein:

FIG. 1 is a schematic illustration of an ultrasound tissue biopsy system provided in accordance with the present disclosure configured for navigation to a target location and for obtaining a tissue sample;

FIG. 2 is a plan view, with parts removed, illustrating a biopsy device of the system of FIG. 1 including angled first and second ultrasound transducers;

FIG. 3 is a side cross-sectional view illustrating the first and second ultrasound transducers of FIG. 2 interfacing with a coupling wedge;

FIGS. 4A and 4B are respective side and front cross-sectional views illustrating the biopsy device of FIG. 2 in a starting condition;

FIG. 5 is a perspective view illustrating a needle cartridge of the biopsy device of FIG. 2 housing a cannula cartridge;

FIGS. 6A-6D are side cross-sectional views of a distal end portion of the biopsy device of FIG. 2 illustrating progressive stages of deployment of a core needle assembly;

FIGS. 7A and 7B are respective side and front cross-sectional views illustrating the biopsy device of FIG. 2 in an armed, pre-fired condition;

FIGS. 8A and 8B are respective side and front cross-sectional views of the biopsy device of FIG. 2 illustrating the needle in a deployed position;

FIGS. 9A and 9B are respective side and front cross-sectional views of the biopsy device of FIG. 2 illustrating the cutting cannula in a deployed position;

FIGS. 10A and 10B are respective side and front cross-sectional views of the biopsy device of FIG. 2 illustrating the core needle assembly in a retracted position;

FIG. 11 is a flow chart of a method for identifying a lesion located within a tissue;

FIG. 12 is a front view of the lesion located within tissue identified by the method of FIG. 11;

FIG. 13 is a front view of sub-regions of the lesion located within the tissue identified by the method of FIG. 11; and

FIG. 14 is a front view of an optimal target of the sub-region of the lesion.

### DETAILED DESCRIPTION

Biopsy devices, biopsy systems, and methods for navigating the biopsy devices to a target location and obtaining a tissue sample using the biopsy device are provided in accordance with the present disclosure and described in detailed below.

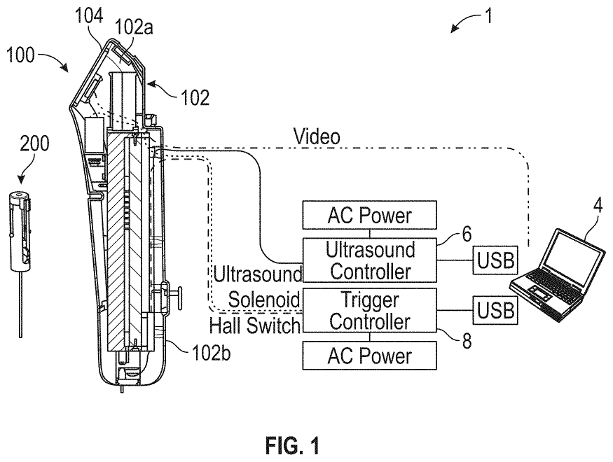

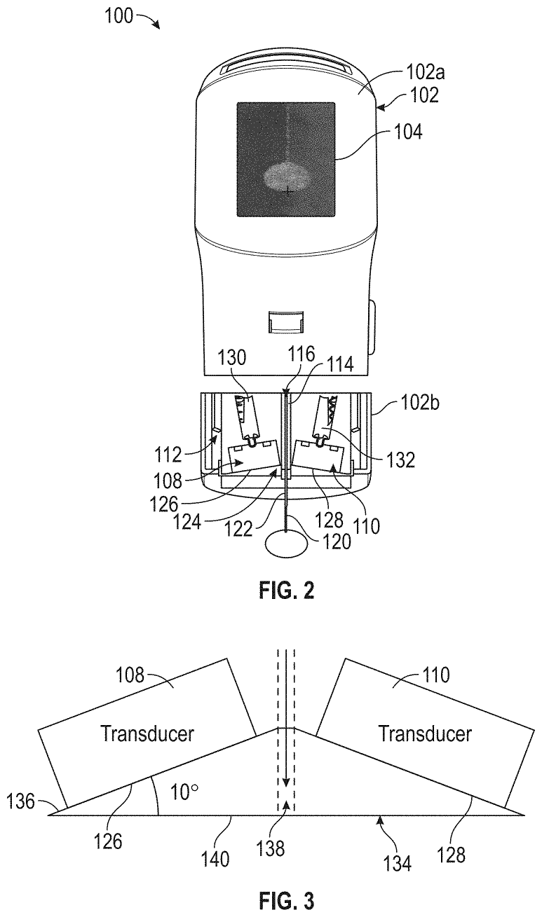

With reference to FIGS. 1-3, an ultrasound tissue biopsy system 1 is provided in accordance with the present disclosure for obtaining a tissue sample from a target tissue, for example, a lesion. The ultrasound tissue biopsy system 1 generally includes a biopsy device 100 incorporating a core needle assembly 200 and a control device 4 (e.g., a computer) interfacing with the biopsy device 100. The biopsy device 100 generally includes an elongated handle body 102, a display 104 supported in a proximal end portion 102a of the handle body 102, and first and second ultrasound transducers 108, 110 supported in a distal end portion 102b of the handle body 102.

The ultrasound transducers 108, 110 are configured to send ultrasound waves toward a selected tissue site, whereby the tissue site, based upon its physical characteristics, reflects ultrasound waves back to the ultrasound transducers 108, 110, which detect the reflected ultrasound waves and send corresponding signals to a processing unit (not shown) of the biopsy device 100. In some aspects, the processing unit may be incorporated into the control device 4. The processing unit is configured to generate an image on the display 104 based upon the signals received from each of the ultrasound transducers 108, 110 by combining the two separate 2D ultrasound images from the ultrasound transducers 108, 110.

The handle body 102 may be fabricated from plastic, such as, for example, PEEK, and defines a hollow interior 112 therein for housing various components of the biopsy device 100. Other suitable materials from which the handle body 102 is formed are contemplated. The handle body 102 may be received in a sterile, disposable cover (not shown). The disposable cover may extend into the handle body 102 to provide a barrier between the handle body 102 and the core needle assembly 200. The handle body 102 may house a memory (e.g., an EEPROM—not shown) for storing a variety of information regarding the biopsy device 100. In embodiments, the memory is additionally or alternatively associated with the control device 100.

The distal end portion 102b of the handle body 102 houses a molded support block 114 therein. The support block 114 defines a longitudinally-extending channel 116 therethrough configured for passage of a needle 120 and a cutting cannula 122 of the core needle assembly 200. The ultrasound transducers 108, 110 may be molded into pockets formed in the support block 114. The ultrasound transducers 108, 110 are set within the support block 114 at an angle relative to one another and laterally spaced from one another to define a space 124 therebetween. As such, the channel 116 of the support block 114 extends between the first and second ultrasound transducers 108, 110, whereby the angled configuration of the first and second ultrasound transducers 108, 110 orients the transducers 108, 110 toward a longitudinal axis defined by the channel 116.

More specifically, each of the first and second ultrasound transducers 108, 110 has a distally-oriented, planar base surface 126, 128 that transmits ultrasound waves therefrom. The base surface 126 of the first transducer 108 defines a plane, and the base surface 128 of the second transducer 110 defines a plane that intersects the plane of the first transducer 108 at an angle of between about 80 degrees and about 170 degrees (wherein “about” takes into account generally accepted tolerances, e.g., material, manufacturing, environmental, measurement, and use tolerances). In embodiments, the angle between the base surfaces 126, 128 may be between about 140 degrees and about 170 degrees, and in some embodiments, about 160 degrees. Each of the transducers 108, 110 has a cable 130, 132 such as, for example, a flex circuit extending therefrom that electrically connects to the processing unit for transmitting electrical signals (e.g., electrical signals representing 2D images) to the processing unit to enable display of an image on display 104 and/or to an ultrasound controller 6 (FIG. 1).

The biopsy device 100 includes a coupling wedge 134 (FIG. 3) interfacing with the base surfaces 126, 128 of the ultrasound transducers 108, 110. Due to the first and second transducers 108, 110 being angled relative to one another and facing the longitudinal axis of the channel 116 of the support block 114, the coupling wedge 134 has a substantially triangular configuration. The coupling wedge 134 is fabricated from an acoustically-transparent material, such as, for example, PEEK, silicone, polyurethane, etc., and has an upper surface 136 that complementarily engages the base surfaces 126, 128 of the respective ultrasound transducers 108, 110. The coupling wedge 134 closes a gap between the bottom surface 126, 128 of the transducers 108, 110 and a skin surface of a patient during use, thereby facilitating the transmission of ultrasound waves from the transducers 108, 110 into tissue.

The coupling wedge 134 defines a channel 138 through a central portion thereof configured for passage of the needle 120 and cutting cannula 122. The channel 138 of the coupling wedge 134 is coaxial with the channel 116 of the support block 114 to allow for the passage of the needle 120 and cutting cannula 122 through the support block 114, the coupling wedge 134, and into tissue. The coupling wedge 134 has a planar, base surface 140 configured to be oriented toward tissue. The base surface 126, 128 of each of the first and second ultrasound transducers 108, 110 is disposed at an acute angle relative to the base surface 140 of the coupling wedge 134. In aspects, the acute angle may be between about 5 degrees and about 20 degrees, and in some embodiments, about 10 degrees. The coupling wedge 134 may be fabricated and subsequently affixed to the transducers 108, 110 or may be molded around the transducers 108, 110.

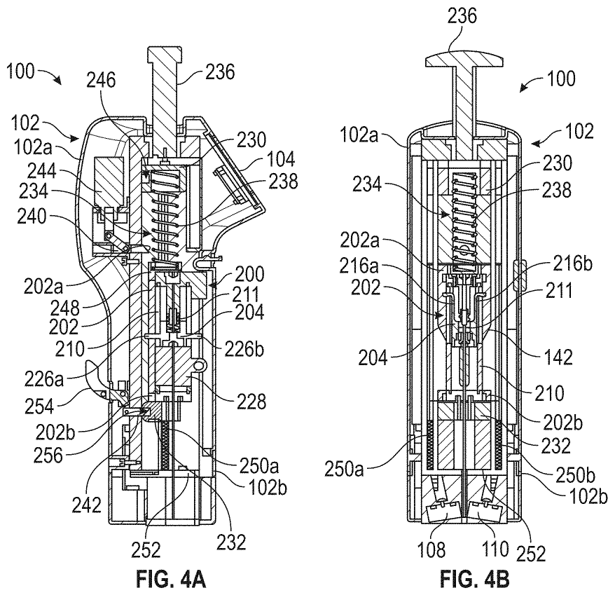

With reference to FIGS. 4A-6D, the core needle assembly 200 of the biopsy device 100 and components for deploying the core needle assembly 200 are now described. The core needle assembly 200 generally includes a needle cartridge 202 and a cannula cartridge 204 movably supported in the needle cartridge 202. The needle 120 is supported by and extends distally from the needle cartridge 202, and the cutting cannula 122 is supported by and extends distally from the cannula cartridge 204.

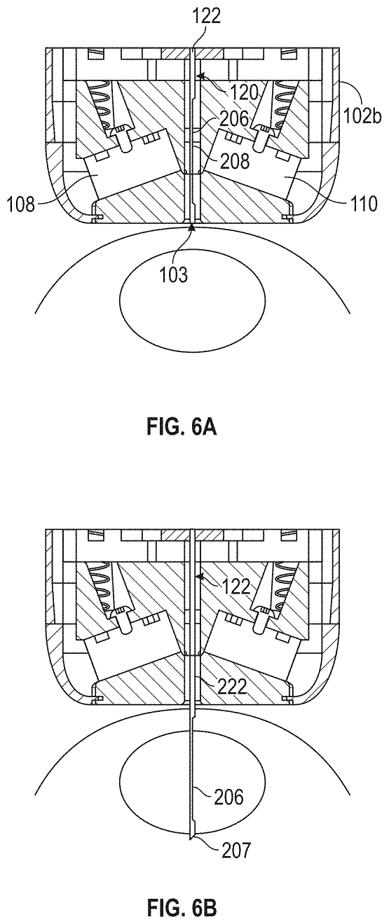

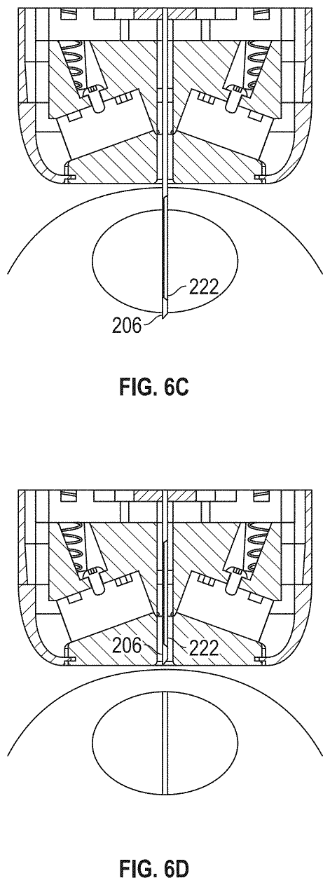

In a starting condition of the biopsy device 100, as shown in FIGS. 4A, 4B, and 6A, a distal tip 206 of the needle 120 is disposed within the distal end portion 102b of the handle body 102 and between the ultrasound transducers 108, 110 in close proximity to an exit opening 103 in the handle body 102. The needle 120 is a side-biting needle defining an elongate cutout 208 in the distal tip 206 configured to receive a sample of tissue therein. In some embodiments, the needle 120 may be a center coring needle or define another suitable configuration. The needle 120 may be echogenically-enhanced to be more visible with ultrasound. For example, the distal tip 206 of the needle 120 may have surface features that are etched (e.g., sandblasted) into the outer surface of the distal tip 206. The surface features may be grooves, notches, or the like.

The needle cartridge 202 includes a hollow cylindrical body 210 slidably supported in the handle body 102. The cylindrical body 210 defines a first pair of longitudinally-extending slots 212 along an outer surface thereof, and a second pair of longitudinally-extending slots 214 along the outer surface thereof (only one of each of slots 212 and 214 is explicitly shown). The cylindrical body 210 defines a ledge 220 disposed at a proximal end of the slot 212 for supporting the cannula cartridge 204 in a retracted position, as will be described.

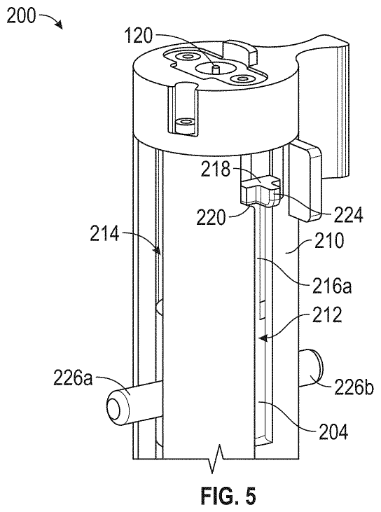

The cannula cartridge 204 is slidably received in the cylindrical body 210 of the needle cartridge 202. A biasing member 211 is disposed within the cylindrical body 210 and between a proximal end portion 202a of the needle cartridge 202 and the cannula cartridge 204. The cannula cartridge 204 has a pair of flexible arms 216a, 216b that extend out of the cylindrical body 210 through the respective first pair of slots 212. Each of the flexible arms 216a, 216b has a T-shaped end 218 (FIG. 5) supported on the ledge 220 of the cylindrical body 210. The ledge 220 of the cylindrical body 210 maintains the cannula cartridge 204 in the retracted position relative to the needle cartridge 202, in which a distal tip 222 of the cutting cannula 122 is spaced proximally from the distal tip 206 of the needle, as shown in FIGS. 6A and 6B.

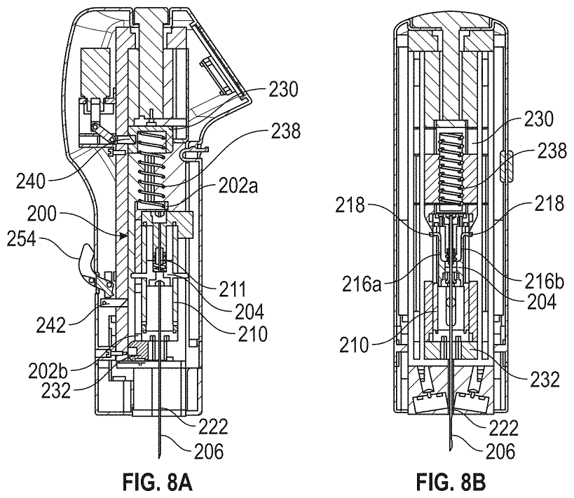

The T-shaped ends 218 of the flexible arms 216a, 216b each have a protrusion 224 disposed radially outward of the respective slot 212 of the cylindrical body 210. The protrusion 224 of each of the T-shaped ends 218 is configured to engage an inner surface 142 of the handle body 102 after the needle cartridge 202 advances toward a deployed position, as shown in FIGS. 6B, 8A, and 8B. The inner surface 142 tapers inwardly in a distal direction, such that upon the T-shaped ends 218 engaging the inner surface 142, the flexible arms 216a, 216b of the cartridge cannula 204 flex inwardly to disengage the T-shaped ends 218 from the supporting ledge 220 of the cylindrical body 210. As such, the distally-oriented bias of the biasing member 211 may then distally urge the cannula cartridge 204 and the attached cutting cannula 122 relative to the needle cartridge 202 toward a deployed position.

The cannula cartridge 204 may include a pair of rigid arms 226a, 226b extending transversely through the second pair of slots 214 in the cylindrical body 210. The rigid arms 226a, 226b may be used to manually retract the cannula cartridge 204 toward the retracted position. The needle core assembly 200 may be shipped with the cutting cannula 122 in the deployed position, as shown in FIGS. 6C and 6D, in which the distal tip 222 of the cutting cannula 122 covers the distal tip 206 of the needle 120. In this way, the distal tip 206 of the needle 120 will be concealed to prevent an accidental piercing. Prior to loading the core needle assembly 200 into the handle body 102, a clinician may grasp the rigid arms 226a, 226b to move the cannula cartridge 204 into the retracted position, thereby loading the biasing member 211. A safety plug 228 may be coupled to the needle cartridge 202 to maintain the cannula cartridge 204 in the retracted position while loading the needle core assembly 200 into the handle body 102. After loading the needle core assembly 200 into the handle body 102, the safety plug 228 may be removed.

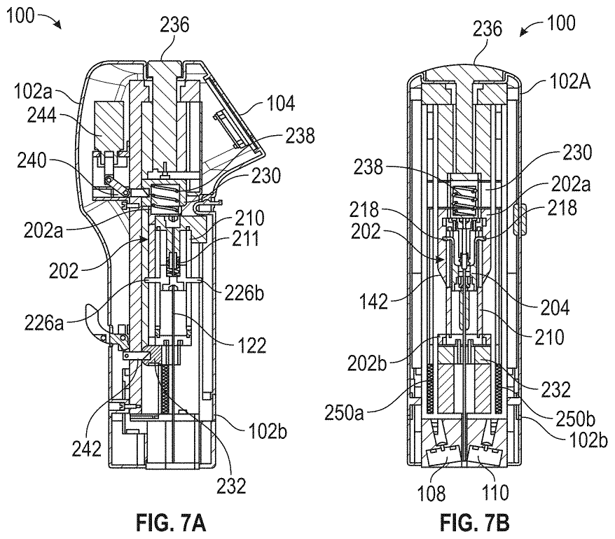

With reference to FIGS. 4A, 4B, 7A, and 7B, the core needle assembly 200 is movably supported in the handle body 102 between a proximal carriage 230 and a distal carriage 232. The proximal and distal carriages 230, 232 and a biasing member 238 (e.g., a coil spring) function together to control a deployment of the needle core assembly 200. The proximal carriage 230 is slidably received in a longitudinally-extending channel 234 in the proximal end portion 102a of the handle body 102. A plunger 236 is coupled to the proximal carriage 230 and extends proximally therefrom and out of an opening in the proximal end portion 102a of the handle body 102 for access by a clinician. The biasing member 238 is disposed between the proximal carriage 230 and the proximal end portion 202a of the needle cartridge 202. A distal translation of the plunger 236 advances the proximal carriage 230 toward the needle cartridge 202, thereby compressing the biasing member 238 to arm the biopsy device 100.

The biopsy device 100 includes a proximal stop 240 disposed in the proximal end portion 102a of the handle body 102, and a distal stop 242 disposed in the distal end portion 102b of the handle body 102. The proximal stop 240 is operably coupled to an actuator, such as, for example, a solenoid 244, and may be configured as a sliding pin extending transversely into the channel 234 of the handle body 102. The proximal carriage 230 defines a recess 246 in a lateral side thereof configured to receive a ramped end surface 248 of the proximal stop 240 upon the proximal carriage 230 moving from a proximal, starting position, shown in FIGS. 4A and 4B, to a distal, armed position, shown in FIGS. 7A and 7B.

Upon advancing the proximal carriage 230 from the starting position to the armed position, the proximal carriage 230 engages the ramped end surface 248 of the proximal stop 240 to shift the proximal stop 240 in a first direction (e.g., to the left in FIG. 4A) out of the path of the proximal carriage 230. When the recess 246 is aligned with the proximal stop 240, the proximal stop 240 shifts in a second direction (e.g., to the right in FIG. 7A) into the recess 246 of the proximal carriage 230 to hold the proximal carriage 230 in a loaded state, in which the biasing member 238 is compressed between the proximal carriage 230 and the proximal end portion 202a of the needle cartridge 202. The solenoid 244 may be in communication (e.g., wireless or wired) with a trigger controller 8 (FIG. 1) for activating the solenoid 244 to move the proximal stop 240 out of engagement with the recess 246 of the proximal carriage 230.

The distal carriage 232 supports a distal end portion 202b of the needle cartridge 202 and is slidably received in the distal end portion 102b of the handle body 102. The distal carriage 232 is resiliently biased toward a proximal position, as shown in FIG. 4A, by a pair of retraction springs 250a, 250b disposed between the distal carriage 232 and a support surface 252 of the handle body 102. The biopsy device 100 has an activation trigger 254 pivotably coupled to the distal end portion 102b of the handle body 102 and accessible by a clinician from outside of the handle body 102. The activation trigger 254 is coupled to the distal stop 242 and is configured to slide the distal stop 242 out of engagement with a recess 256 defined in a lateral side of the distal carriage 232.

When the distal stop 242 is disposed within the recess 256 of the distal carriage 232, the distal carriage 232 is prevented from advancing from the proximal position as the proximal carriage 230 is advanced from the proximal position (FIGS. 4A and 4B) to the distal position (FIGS. 7A and 7B). In this way, during advancement of the proximal carriage 230, the biasing member 238 is compressed between the proximal carriage 230 and the proximal end portion 202 of the needle cartridge 202 so long as the needle cartridge 202 is held in position by the distal carriage 232. With the proximal carriage 230 fixed in the distal position by the proximal stop 240, a release of the distal carriage 232 from the distal stop 242 allows the biasing member 238 to distally urge the needle cartridge 202, which carries along therewith the needle 120, the cannula cartridge 204, and the cutting cannula 122.

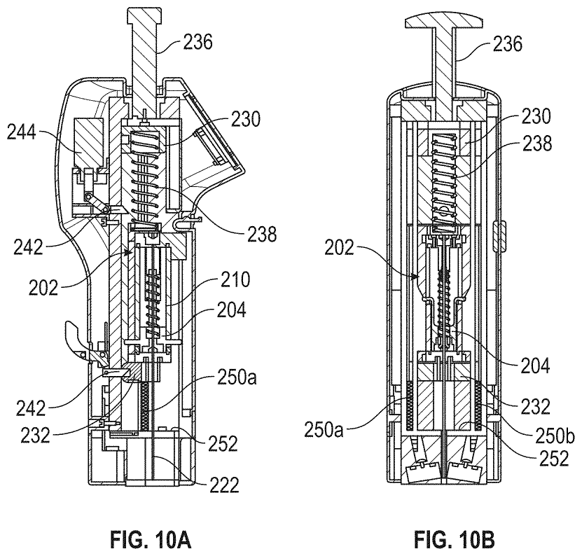

One use of the ultrasound tissue biopsy system 1 for extracting tissue samples from a lesion, e.g., a tumor, is described in detail with reference to FIGS. 7A-10B. The biopsy device 100 is positioned such that the distal end portion 102b of the handle body 102 is placed in abutting engagement with an outer surface of tissue (e.g., breast tissue), with the distal tip 206 of the needle 120 in proximity to target tissue, e.g., a lesion. The ultrasound transducers 108, 110 emit ultrasound waves toward the lesion and the distal tip 206 of the needle 120. The ultrasound transducers 108, 110 then receive the reflected ultrasound waves and communicate signals corresponding to the same to the processing unit (not shown) of the computer 4 which generates an image of the distal tip 206 of the needle 120 relative to the lesion on the display 104. In addition, the processing unit of the system 1 may animate a projected needle pathway on the display 104 such that a clinician can accurately predict the pathway the needle 120 will travel if actuated at the present position. The biopsy device 100 is moved to a position in which the projected needle pathway animated on the display 104 is aligned with the image of the lesion.

Upon aligning the projected needle pathway with the displayed image of the lesion on the display 104, the plunger 236 of the biopsy device 100 is actuated. With the distal carriage 232 held in the proximal position by the distal stop 242, advancement of the plunger 236 compresses the biasing member 238 between the proximal and distal carriages 230, 232, thereby arming the biopsy device 100, as shown in FIGS. 7A and 7B. To actuate the core needle assembly 200, the activation trigger 254 is actuated (e.g., pivoted) to disengage the distal stop 242 from the distal carriage 232, whereby the biasing member 238 urges the needle 120 from the retracted position (FIGS. 6A, 7A, and 7B) toward the deployed position (FIGS. 6B, 8A, and 8B) and into the lesion. Since the cannula cartridge 204 of the core needle assembly 200 is supported in the needle cartridge 202, the cannula cartridge 204 and the cutting cannula 122 advance with the needle cartridge 202 and needle 120.

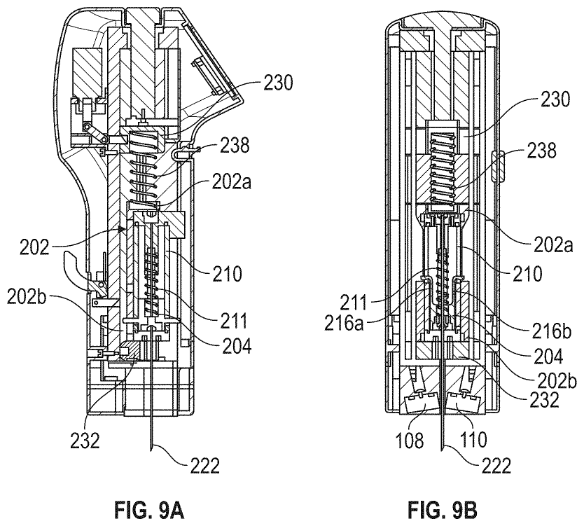

With reference to FIGS. 8A and 8B, upon the needle 120 advancing to the deployed position, or any suitable advanced distance, the protrusions 224 of the flexible arms 216a, 216b of the cannula cartridge 204 engage the tapered inner surface 142 of the handle body 102, whereby the T-shaped ends 218 of the flexible arms 216a, 216b disengage the ledge 220 of the cylindrical body 220. With the flexible arms 216a, 216b of the cannula cartridge 204 no longer being supported on the ledge 220 in the retracted position, the biasing member 211 in the cylindrical body 210 urges the cutting cannula 122 axially relative to the needle 120 to the deployed position (FIGS. 9A and 9B), in which the distal tip 222 of the cutting cannula 122 is disposed over the distal tip 206 of the needle 120 and extends distally beyond the distal end portion 102b of the handle body 102. With the distal tip 222 of the cutting cannula 122 encircling the distal tip 206 of the needle 120, the sample of tissue is captured in the elongate cutout 208 in the needle 120, as shown in FIG. 6C.

During advancement of the needle 120, the distal tip 206 of the needle 120 may naturally deflect in a direction away from a beveled edge 207 (FIG. 6B) that forms the piercing edge of the distal tip 206. As such, to ensure that the distal tip 206 remains within the imaging plane of the ultrasound transducers 108, 110, the angular orientation of the needle 120 is such that a plane defined by the beveled edge 207 of the distal tip 206 is perpendicular relative to the imaging plane defined by the ultrasound transducers 108, 110. In this way, as the needle 120 is advanced through tissue, the distal tip 206 will deflect in a direction parallel with the imaging plane so as to remain visible by the ultrasound transducers 108, 110 as opposed to deflecting outside of the imaging plane.

Upon advancing the cutting cannula 122 to the deployed position, the solenoid 244 may be configured to automatically retract the proximal stop 240 to release the proximal carriage 230 from the proximal stop 240. Releasing the proximal carriage 230 from the proximal stop 240 allows the retraction springs 250a, 250b and the biasing member 238 to retract the needle core assembly 200 out of the tissue and back into the handle body 102. In embodiments, actuation of the solenoid 244 may be delayed for a selected amount of time after deployment of the cutting cannula 122 to allow for an image capture of the needle 120 and cutting cannula 122 prior to their withdrawal from the tissue, thus allowing for visual confirmation of the capture of the tissue sample. A magnet (not explicitly shown) may be incorporated into the distal carriage 232 and positioned for sensing by a hall effect sensor (not explicitly shown) in the handle body 102 to determine when the cutting cannula 122 has entered the deployed position. Other types of sensors may be provided, and at any suitable locations of the biopsy device 100, to detect one or more stages of deployment of the needle core assembly 200.

In embodiments, the biopsy device 100 may be configured to deploy a marker or tracking device therefrom in addition to or instead of the core needle assembly 200. The marker may be a wire or rod that extends from the patient's skin or may be a small implantable device or chip. A cartridge housing with the marker may be loaded into the biopsy device 100 and deployed therefrom. In other embodiments, the core needle assembly 200 may further include a marker and a mechanism for deploying the marker simultaneously with deployment of the needle 120 and cutting cannula 122.

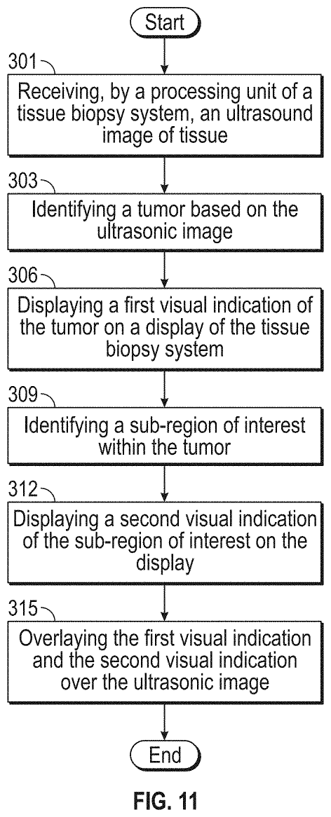

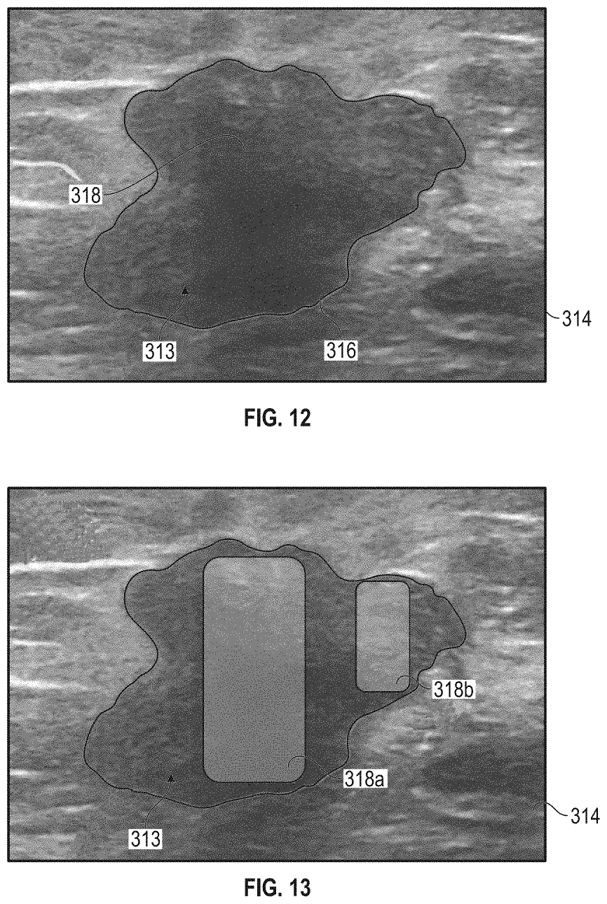

With reference to FIGS. 11-14, the controller 6 (FIG. 1) and/or processing unit detailed above may be further configured to identify and display on the display 104 a tumor, such as, for example, a lesion 313 (FIG. 12) located within tissue imaged by biopsy device 100. In particular, with reference to FIG. 11, in step 301, the processing unit 6 (FIG. 1), for example, receives signals from each of the ultrasound transducers 108, 110 and combines the two separate 2D ultrasound images to produce a raw ultrasound image 314. In step 303, the processing unit 6 identifies the lesion 313 based on the raw ultrasound image 314.

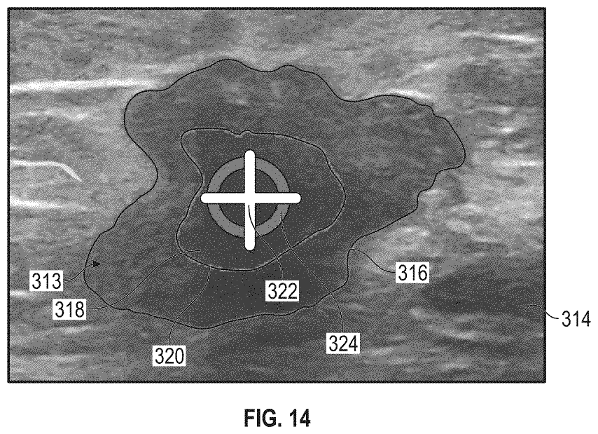

To identify the lesion 313 located within the tissue, the processing unit 6 applies a radiomic transformation on the raw ultrasound image 314. The processing unit 6 may further apply a classification algorithm on the raw ultrasound image 314 including the identified lesion 313 to classify the identified lesion 313. In step 306, the processing unit 6 displays a first visual indication of the identified lesion 313 on the display 104. To display the first visual indication of the identified lesion 313, the processing unit 6 applies segmentation algorithm to identify the boundaries of the identified lesion 313. The first visual indication may include displaying a line 316 (FIGS. 12 and 14) defining the boundary of the identified lesion 313.

In step 309, the processing unit 6 may identify a sub-region of interest 318 within the identified lesion 313. To identify the sub-region of interest 318 within the identified lesion 313, the processing unit 6 applies additional radiomic transformations on the identified lesion 313. The tissue type of the sub-region of interest 318 within the identified lesion 313 may be an active sub-region 318a or a necrotic sub-region 318b. In step 312, the processing unit 6 displays a second visual indication of the type of sub-region of interest 318 within the identified lesion 313 on the display 104. To display the second visual indication of sub-region of interest 318, the processing unit 6 applies additional segmentation and classification algorithm(s) to the identified lesion 313 to identify the boundaries of the sub-region of interest 318 within the identified lesion 313. In some embodiments, the identified lesion 313 may include multiple sub-regions and/or sub-regions of interest. The second visual indication may include displaying a line 320 (FIG. 14) defining the boundary of the sub-region of interest 318 within the identified lesion 313. In another aspect, as shown in FIG. 13, displaying the second visual indication may include highlighting each type of sub-region of interest within the identified lesion 313 and/or applying shading (and, for multiple sub-regions, discrete shading) to each type of sub-region of interest within the identified lesion 313.

In step 315, to display the first visual indication and the second visual indication on the display 104, the first visual indication and the second visual indication may be overlaid over the raw ultrasound image 314. In some embodiments, the processing unit 6 may optionally display the first visual indication or the second visual indication. The first visual indication and the second visual indication may be displayed in real time and overlaid over real-time ultrasound image 314. In some embodiments, the first visual indication and the second indication may be selectively toggled on or off on the display 104. Alternatively, the first visual indication and the second indication may be automatically displayed on the display 104 based on the detections of the lesion 313.

With reference to FIG. 14, in some embodiments, the processing unit 6 may be further configured to display a third visual indication, such as, for example, an optimal target location 322 of the sub-region of interest 318 of the identified lesion 313, such that a clinician is given a visual display of the precise location where a biopsy should be taken. The optimal target location 322 maximizes the probability of obtaining an acceptable histological sample of a lesion's sub-region of interest 318 obtained by inserting the biopsy device 100 into a central location of the lesion 313. To determine the optimal target location 322, the processing unit 6 may apply an optimization algorithm. The third visual indication may include displaying a line 324 around the boundaries of the optimal target location 322 within the sub-region of interest 318. The display 104 may also display cross-hairs within the line 124 to pinpoint the center of the optical target location 322. The processing unit 6 may further display other related diagnostic data.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A tissue biopsy system, comprising:
a biopsy device including:
a handle body;
at least one transducer disposed within the handle body; and
a needle configured to be deployed from the handle body and into tissue;
a display configured to display an ultrasound image of the tissue based on data collected by the at least one transducer; and
a processing unit in communication with the biopsy device and the display, the processing unit configured to:
identify an area of interest, located in the tissue, based on the ultrasound image; and
visually indicate the area of interest on the display.

2. The tissue biopsy system according to claim 1, wherein the display is in operable communication with the processing unit.

3. The tissue biopsy system according to claim 2, wherein the display forms a proximal end portion of the handle body.

4. The tissue biopsy system according to claim 2, wherein the display is remote from the handle body and coupled to a proximal end portion of the handle body.

5. The tissue biopsy system according to claim 1, wherein in identifying the area of interest, the processing unit applies at least one radiomic transformation on the ultrasound image, processes the transformed image with a segmentation and/or classification algorithm, and visually indicates the area of interest by displaying a line defining a border of the area of interest on the display.

6. The tissue biopsy system according to claim 1, wherein the processing unit is further configured to identify a sub-region of interest within the area of interest.

7. The tissue biopsy system according to claim 6, wherein in identifying the sub-region of interest, the processing unit applies at least one radiomic transformation on the ultrasound image, processes the transformed image with a segmentation and/or classification algorithm, and provides a visual indication of the sub-region of interest, and
wherein the visual indication of the sub-region of interest includes displaying a line defining a border of the sub-region of interest.

8. The tissue biopsy system according to claim 7, wherein the sub-region of interest is at least one of a necrotic sub-region or an active sub-region.

9. The tissue biopsy system according to claim 7, wherein the visual indication of the sub-region of interest further includes at least one of highlighting sub-regions of interest or applying discrete shading to each type of sub-region of interest.

10. The tissue biopsy system according to claim 7, wherein the processing unit is further configured to identify a biopsy-target area within the active sub-region and visually indicate the biopsy-target area on the display.

11. The tissue biopsy system according to claim 7, wherein the processing unit is further configured to overlay the visual indication of the sub-region of interest over the ultrasound image on the display.

12. The tissue biopsy system according to claim 1, wherein the area of interest is a tumor.

13. A method of performing a tissue biopsy, the method comprising:
receiving, by a processing unit of a tissue biopsy system, an ultrasound image of tissue;
identifying an area of interest based on the ultrasound image; and
displaying a first visual indication of the area of interest on a display of the tissue biopsy system.

14. The method according to claim 13, wherein identifying the area of interest includes applying at least one radiomic transformation on the ultrasound image, and processing the transformed image with a segmentation and/or classification algorithm.

15. The method according to claim 13, wherein displaying the first visual indication includes displaying a line defining a border of the area of interest.

16. The method according to claim 13, further comprising:
identifying a sub-region of interest within the area of interest; and
displaying a second visual indication of the sub-region of interest on the display.

17. The method according to claim 16, wherein identifying the sub-region of interest within the area of interest includes applying at least one radiomic transformation on the ultrasound image, and processing the transformed image with a segmentation and/or classification algorithm.

18. The method according to claim 16, wherein the sub-region of interest within the area of interest is at least one of a necrotic sub-region or an active sub-region.

19. The method according to claim 16, wherein displaying the first visual indication includes displaying a line defining a border of the area of interest, and displaying the second visual indication includes displaying a line defining a border of the sub-region of interest.

20. The method according to claim 16, wherein displaying the second visual indication includes at least one of highlighting the sub-region of interest or applying discrete shading to each type of sub-region of interest.

21. The method according to claim 16, further comprising overlaying the first visual indication and the second visual indication over the ultrasound image.

22. The method according to claim 13, wherein the area of interest is a tumor.
